(19) United States
(12) Patent Application Publication
(10) Pub. No.: US 2010/0131032 A1
(43) Pub. Date: May 27, 2010

(54) Audio Prostheses Product Selection

(75) Inventors: Manuela Oberhofer, Innsbruck (AT); Ute Hellmuth-Zweyer, Innsbruck (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/623,649

(22) Filed: Nov. 23, 2009

Related U.S. Application Data
(60) Provisional application No. 61/118,040, filed on Nov. 26, 2008.

(51) Int. Cl. A61F 11/04; A61N 1/36

(52) U.S. Cl. 607/57

(57) ABSTRACT

Selection of a prosthetic hearing product is described. A patient testing module tests hearing characteristics of a potential patient to determine a patient audiogram representing patient hearing impairment. A product comparison module compares the patient audiogram to a plurality of product audiograms, where each product audiogram represents a region of hearing impairment treatable by a specific prosthetic hearing product. A product selection module selects a specific prosthetic hearing product for the potential patient based on the audiogram comparison.

This application claims priority from U.S. Provisional Patent Application 61/118,040, filed Nov. 26, 2008, which is incorporated herein by reference.

### FIELD OF THE INVENTION

The present invention relates to medical implants, and more specifically to techniques for selecting between different types of prosthetic hearing implant products.

### BACKGROUND ART

A normal ear transmits sounds as shown in FIG. 1 through the outer ear 101 to the tympanic membrane (eardrum) 102, which moves the ossicles of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

When this arrangement does not function properly, hearing is impaired. Audiologic advancements in hearing assessment and diagnosis of hearing disorders has resulted in better diagnosis and treatment for the hearing impaired. State regulation of the dispensing industry through licensing and certification programs also was developed to insure quality of hearing aid dispensing practices.

Current prosthetic hearing products have improved signal processing circuits and enhanced fitting parameters that allow them to be customized to the patient's individual hearing impairment (similar to an eye glass prescription, one size does not fit all). Some new products can be located entirely in the patient's ear canal and are cosmetically superior to the large bulky devices of years past. Many manufacturers participate in the prosthetic hearing marketplace which is a sizable 3 billion dollar worldwide market. At present, a hearing impaired patient has a wide variety of prosthetic hearing products to choose between.

One type of prosthetic hearing product is the conventional hearing aid where a microphone detects sound which is amplified and transmitted in the form of acoustical energy by a speaker or another type of transducer into the middle ear 103 by way of the tympanic membrane 102. Interaction between the microphone and the speaker can sometimes cause an annoying and painful a high-pitched feedback whistle. The amplified sound produced by conventional hearing aids often also includes a significant amount of distortion.

For many patients with severe to profound hearing impairment, cochlear implants can evoke auditory sensations by electrical stimulation of the cochlea 104. FIG. 1 shows some components of a typical cochlear implant system. An external microphone provides an audio signal input to an external signal processing stage (not shown in FIG. 1) where various signal processing schemes can be implemented. The processed signal is then converted into a digital data format, such as a sequence of data frames, for transmission into receiver stimulator 108. Besides extracting the audio information, the receiver stimulator 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Besides cochlear implant systems as such, some patients with some residual hearing (partial deafness) are now benefiting from hybrid electric and acoustic stimulation (EAS) devices as first described in von Ilberg et al., Electric-Acoustic Stimulation Of The Auditory System, ORL 61:334-340 (1999), which is incorporated herein by reference. EAS products combine the use of a conventional hearing aid (HA) device to provide acoustic-mechanical stimulation of lower audio frequencies to the patient's ear drum and a cochlear implant (CI) to provide intracochlear electrical stimulation of higher audio frequencies to the auditory nerve. For example, see Lorens et al., Outcomes Of Treatment Of Partial Deafness With Cochlear Implantation: A DUET Study, Laryngoscope, 2008 Feb.: 118(2):288-94, which is incorporated herein by reference.

Direct-drive implantable middle ear hearing devices represent another new category of prosthetic hearing products. Rather than delivering acoustic energy into the external auditory canal (as with conventional hearing aids), direct drive middle ear implant systems use mechanical vibrations delivered directly to the ossicular chain, while leaving the ear canal completely open. For example, the Vibrant-Med-El Soundbridge® has two major components: 1) an implant called the Vibrating Ossicular Prosthesis (VORP) which uses a Floating Mass Transducer (FMT) to stimulate the ossicles, and 2) an external digital audio processor that is programmed by an audiologist or hearing aid acoustician several weeks after the implant procedure to fit the user's specific hearing impairment.

### SUMMARY OF THE INVENTION

Embodiments of the present invention include systems, methods, and computer program products for selection of a prosthetic hearing product. A patient testing module tests hearing characteristics of a potential patient to determine a patient audiogram representing patient hearing impairment. A product comparison module compares the patient audiogram to a plurality of product audiograms, where each product audiogram represents a region of hearing impairment treatable by a specific prosthetic hearing product. A product selection module selects a specific prosthetic hearing product for the potential patient based on the audiogram comparison.

In some specific embodiments, the prosthetic hearing products may include at least one cochlear implant system, which may have different cochlear implant options, electrode array options, and/or external processor options. The prosthetic hearing products may include at least one hybrid electric acoustic implant system, for example, with different cochlear implant options. The prosthetic hearing products may include at least one middle ear implant system, for example, with different middle ear implant options.

The product selection module may further consider additional selection criteria together with the audiograms for selecting a specific prosthetic hearing product. These additional selection criteria may include past medical history of the potential patient, speech scores representing word and/or syllable comprehension testing results, and/or an overall impairment value representing severity of hearing impairment in the potential patient.

Embodiments may also include an interactive user interface for displaying a representation of the product selection process. For example, the interactive user interface may display the patient audiogram and one or more of the product audiograms, which may usefully be color coded.

### BRIEF DESCRIPTION OF THE DRAWINGS

FIG. 1 shows structures of a typical ear which includes a cochlear implant.

FIG. 2 shows a specific example of a prosthetic hearing product selection system according to one embodiment of the present invention.

FIG. 3 shows various logical steps in a corresponding method of selecting a prosthetic hearing product.

FIG. 4A-D shows examples of various different patient audiograms.

FIG. 5 shows an example of a product audiogram according to one embodiment of the present invention.

FIG. 6 shows another example of a product audiogram according to one embodiment of the present invention.

FIG. 7 shows another example of a product audiogram according to one embodiment of the present invention.

FIG. 8 shows another example of a product audiogram according to one embodiment of the present invention.

FIG. 9 shows an example of multiple product audiograms according to one embodiment of the present invention.

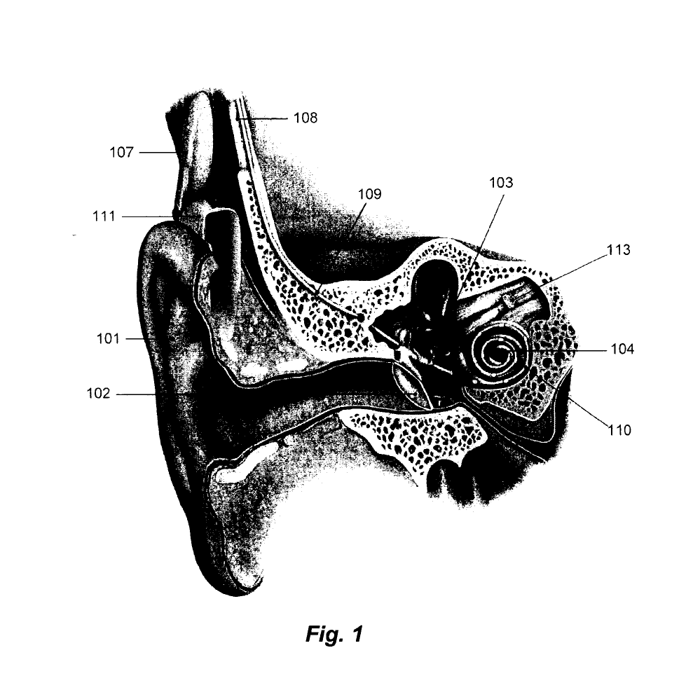

### DETAILED DESCRIPTION OF SPECIFIC EMBODIMENTS

Until now, selection of an prosthetic hearing product for a given patient has been a custom process performed by a trained audiologist based on their professional evaluation of the patient's hearing impairment and their own knowledge of existing prosthetic hearing products. Embodiments of the present invention define, structure, and automate the process for selecting a prosthetic hearing product for a given patient.

Such embodiments may be implemented at Comprehensive Hearing Implant Centers at various convenient locations. In addition to evaluating and selecting prosthetic hearing products for potential patients, some or all such Comprehensive Hearing Implant Centers also may provide the capability and knowledge to implant and fit some or all such products. Such selection is based on a comparison of a measured audiogram of a potential patient, by the audiologist, surgeon or even the patient himself with existing product characteristic audiograms to find the most appropriate prosthetic hearing product solution. Typically, this involves use of an electronic presentation such as an interactive computer display, although conceivably, the principles could extend to use in other specific forms such as a part of a slide show presentation or in printed form such as where product audiogram transparencies overlay each other with each transparency highlighting one of the characteristic areas for a given prosthetic hearing product solution.

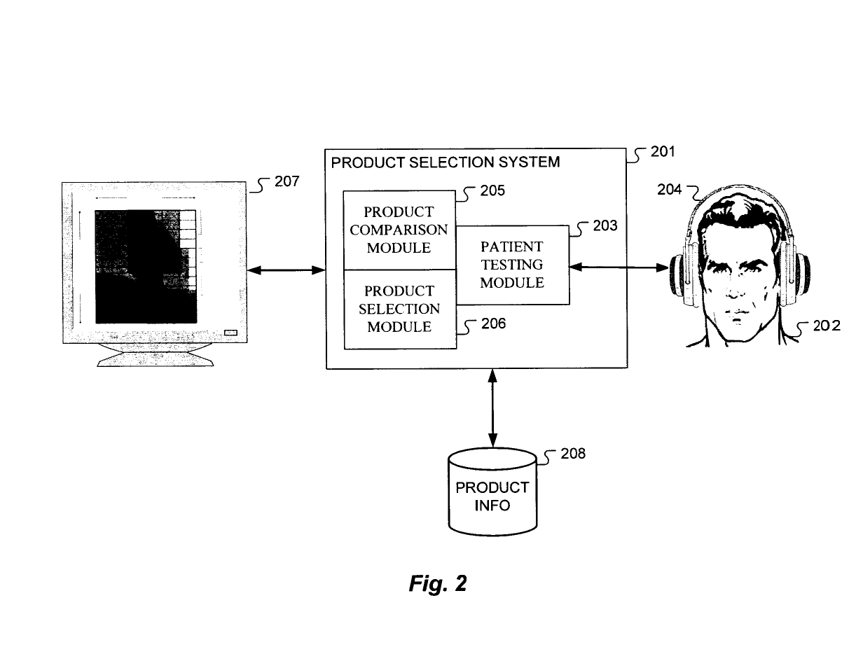

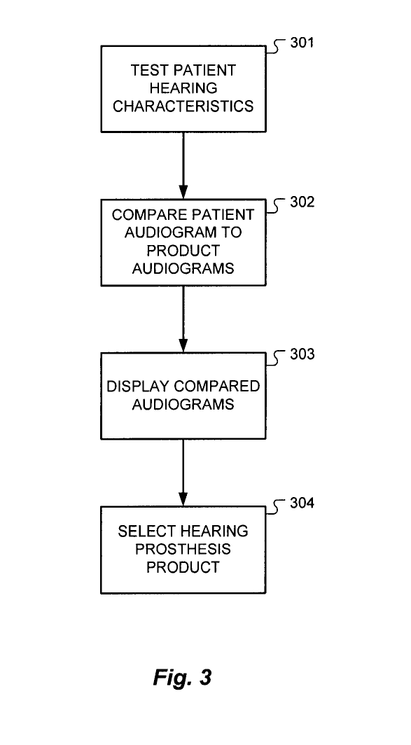

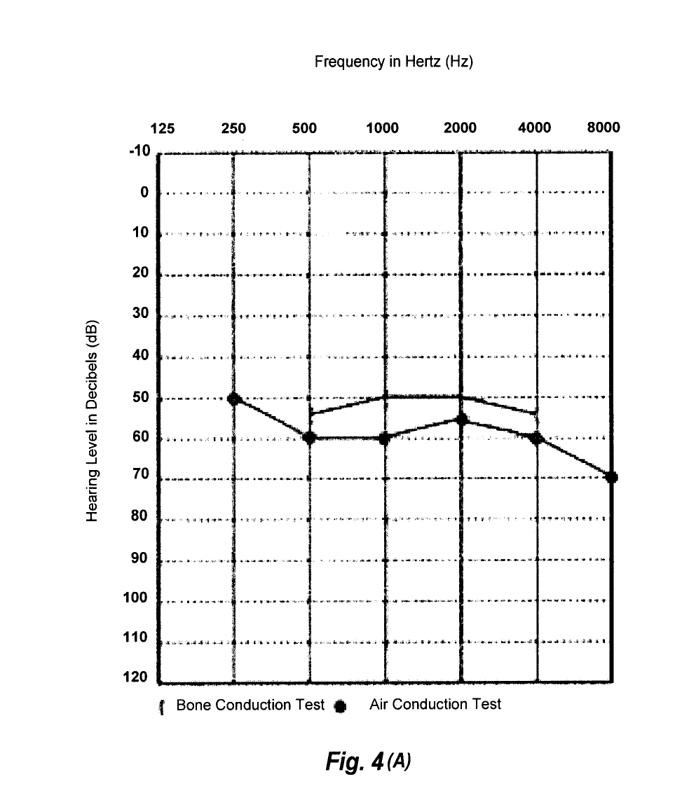

FIG. 2 shows a specific example of a prosthetic hearing product selection system according to one embodiment of the present invention. FIG. 3 shows various logical steps in a corresponding method of selecting a prosthetic hearing product. A product selection system 201 includes a patient testing module 203 in bi-directional communication with the patient 202 being evaluated.

Initially, the patient testing module 203 tests the patient's hearing characteristics, step 301, to develop a patient audiogram that represents the specific characteristics of the hearing impairment of the patient 202. For example, one widely used testing approach is known as Pure Tone Audiometry which uses pure tone signals at various audio frequencies and variable sound intensities ranging from −10 dB to +120 dB. Signals of decreasing intensity at each frequency are presented to the patient 202 to determine an intensity level at which they cannot hear the test tone. A patient audiogram plots the hearing threshold levels at each frequency. The patient testing module 203 may assess multiple specific hearing characteristics of the patient 202. For example, the functioning of both the conduction (outer and middle ear) components and the sensorineural (cochlea and auditory nerve) components of the auditory system is measured by what is referred to as air conduction. The patient 202 wears headphones 204 and the signal passes by air conduction through the outer and middle ear to the inner ear, auditory nerve and auditory cortex of the brain. The headphones 204 can also be used to apply a vibratory stimulus to the skull of the patient 202 to directly stimulate the cochlea by bone conduction to assess the function of the cochlea and auditory nerve. By using both air conduction measurements and bone conduction measurements, the type of hearing impairment of the patient 202 can be classified as conductive, sensorineural, or mixed type.

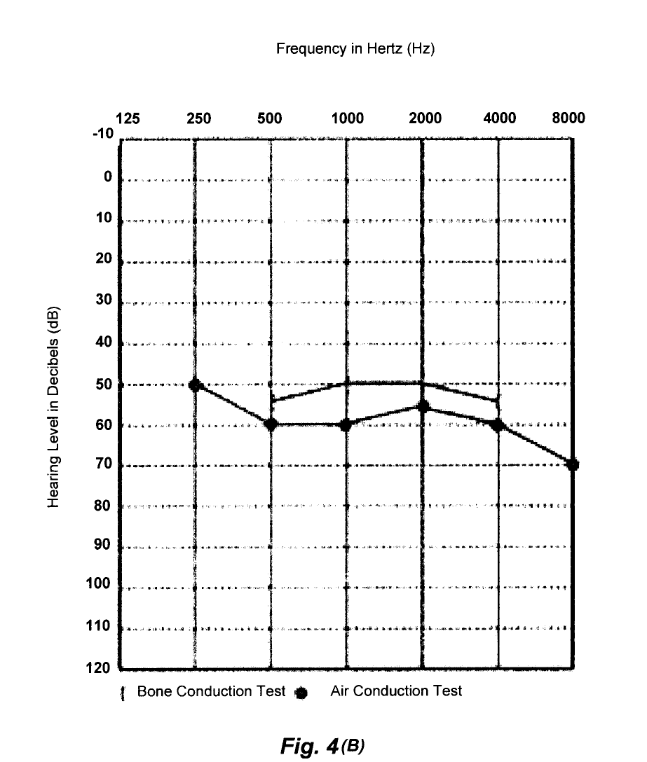

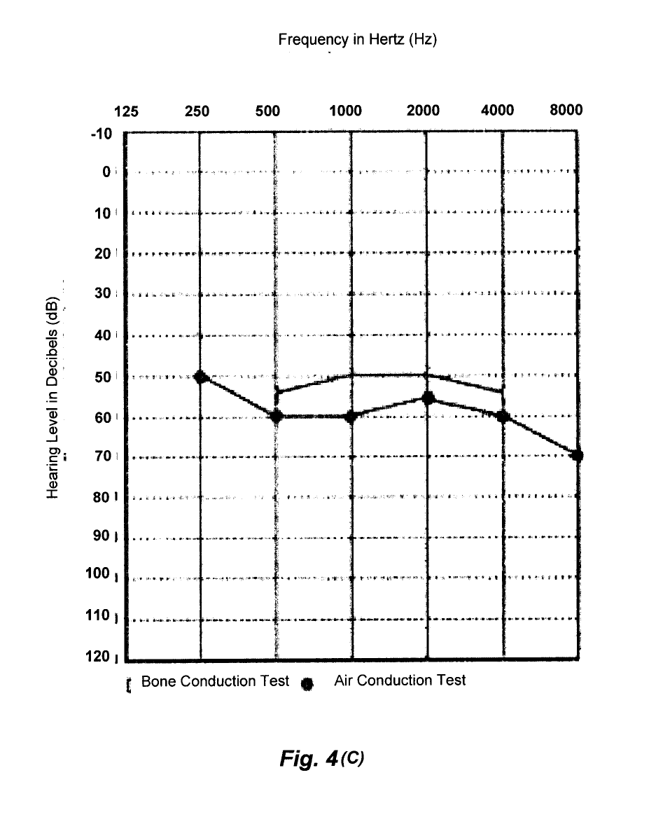

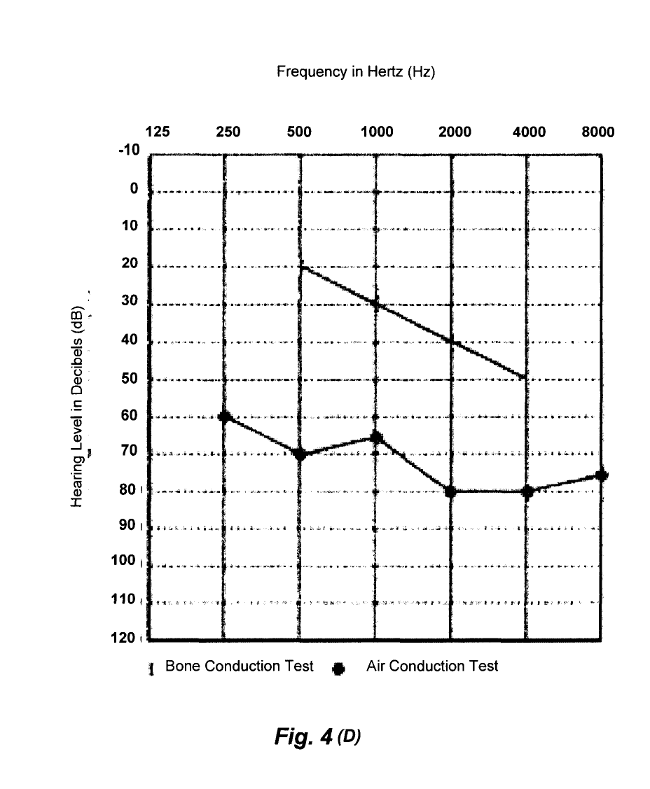

FIG. 4A-D shows various examples of patient audiograms. FIG. 4A shows a normal audiogram—a normally hearing person would expect to have a hearing threshold measurement of 20 dB or better, and this represents no hearing impairment on the audiogram. FIG. 4B is an example of an audiogram showing conductive hearing impairment in the left ear. In this example, the upper line shows the results of bone conduction measurements and the lower line shows air conduction measurements. This gap between the air conduction and bone conduction lines indicates a conductive hearing problem in either the external or middle ear. FIG. 4C is an example of an audiogram of the right ear where the bone conduction line and the air conduction line are similar, indicating sensorineural hearing impairment where the impairment is in the cochlea or auditory nerve. FIG. 4D shows an audiogram of the right ear exhibiting mixed hearing impairment where both the air conduction and bone conduction measurements are reduced from normal hearing. From one specific embodiment to another there may be minor differences in the specific scaling of the audiograms.

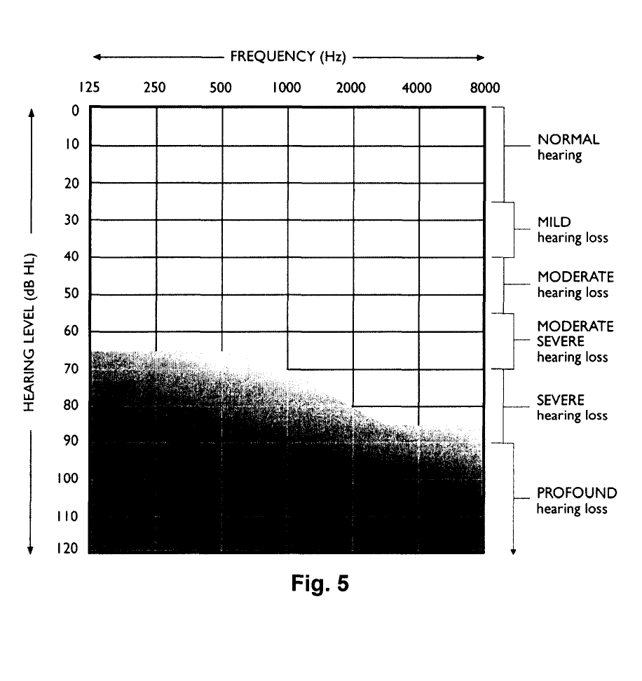

Returning to FIGS. 2 and 3, a product comparison module 205 compares the patient audiogram to a plurality of product audiograms stored in a product information database 208, step 302. Each product audiogram represents a region of hearing impairment that is treatable by a specific prosthetic hearing product, where the different product audiogram regions may be color coded. Examples of such product audiograms are depicted in FIGS. 5-8. A product selection module 206 selects a specific prosthetic hearing product, step 304, for the potential patient based on the audiogram comparison. The product selection module 206 may also take into account additional selection criteria together with the audiograms for selecting a specific prosthetic hearing product. Examples of such additional selection criteria include without limitation past medical history of the potential patient, speech scores representing word comprehension testing results, speech scores representing syllable comprehension testing results, and an overall impairment value representing severity of hearing impairment in the potential patient.

For example, for individuals having patient audiograms indicating severe to profound hearing impairment, and who obtain little or no benefit from acoustic amplification may benefit from a prosthetic hearing product in the specific form of a cochlear implant system such as Med-El's MAESTRO™ product. Such individuals would have patient audiograms in the lower portion of the frequency-intensity plot corresponding to the shaded region shown in FIG. 5, which also is the product audiogram for a cochlear implant system. Besides the patient audiogram and the various product audiograms, examples of additional selection criteria that may be considered with regards to selecting a specific cochlear implant system may include a requirement that the patient has a functional auditory nerve and be preoperatively assessed and screened. Except for circumstances when it is not an option (such as hearing impairment due to infectious disease), potential patients also may be required to undergo a trial period with a conventional acoustic mechanical hearing aid before a cochlear implant system is selected. Candidate patients also should be sufficiently motivated, realistic about the benefits of cochlear implantation, and be sufficiently educated about participation in regular audio processor programming, assessment, and training.

In the specific case of a cochlear implant product, a potential patient can be evaluated to select among multiple specific implant designs, electrode arrays, audio processor designs and wearing options for the external audio processors. And again, this may take into account both comparison of the patient audiogram with product audiograms as well as additional selection criteria. For example, the condition of the patient's cochlea can affect the best specific type of electrode array for use in the cochlear implant system. A standard electrode array may be selected for insertion into an open cochlea and stimulation of the complete audio frequency range. Alternatively, a special soft and flexible electrode may be selected where there is a greater need for atraumatic insertion such as into an open cochlea. Or in an open cochlea where complete cochlear coverage is not available or not desirable, a medium length electrode array may be selected. A compressed electrode may be selected for insertion into a cochlea with moderate obliteration, ossification, or malformation, while a split branch electrode may be selected for insertion into a cochlea with severe obliteration or ossification.

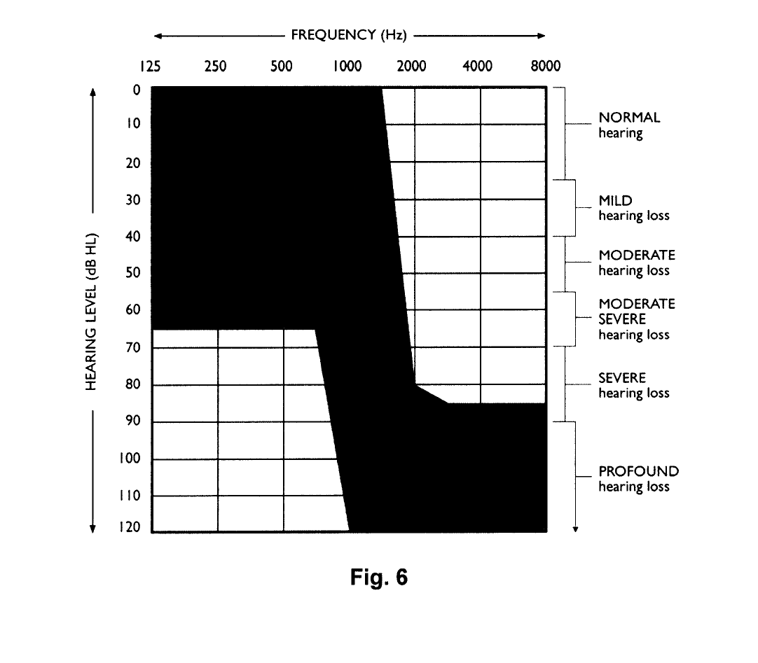

For a patient audiogram indicating mild to moderate low frequency sensorineural hearing impairment which slopes towards a more profound hearing impairment in the higher frequencies, such as the range highlighted in FIG. 6, a hybrid electric acoustic prosthetic hearing product such as the Med-El EAS™ Hearing System can be a useful solution. Such a system typically includes a cochlear implant system as described above (e.g., Med-El PULSAR_{CI}¹⁰⁰ and SONATA_{TI}¹⁰⁰ cochlear implants and electrode arrays such as the Med-El FLEX^{EAS} electrode array) as well as various external audio processors such as the Med-El DUET™ family of audio processors.

As with selection of other specific prosthetic hearing products, this may take into account both comparison of the patient audiogram with product audiograms as well as additional selection criteria. For example, pure tone threshold indications for a hybrid electric acoustic product are a mild to moderate low frequency sensorineural hearing impairment, sloping to a profound hearing impairment in the higher frequencies, with speech word comprehension scores ≦60% at 65 dBSPL in the best aided condition. Other embodiments may use syllable comprehension scores in addition to or instead of word comprehension scores. Examples of other additional selection criteria may include that the prospective patient suffers no progressive hearing impairment, no autoimmune disease, no hearing impairment as a result of meningitis, otosclerosis or ossification, no malformations or obstruction of the cochlea, no air-bone gap >15 dB, and/or no external ear contra-indications to using amplification devices. And as with cochlear implant systems generally, potential patients may be required to undergo a trial period with a conventional acoustic mechanical hearing aid before a cochlear implant system is selected. Candidate patients also should be sufficiently motivated, realistic about the benefits of cochlear implantation, and be sufficiently educated about participation in regular audio processor programming, assessment, and training

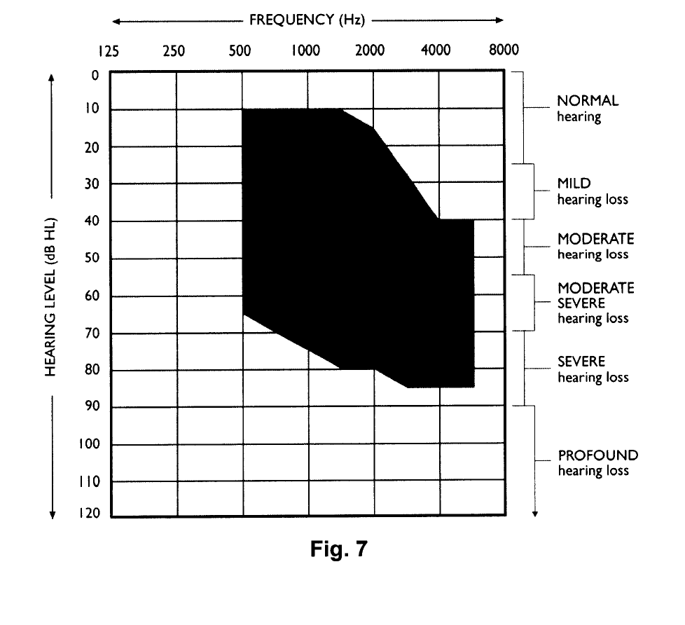

For patient audiograms indicating mild to severe sensorineural hearing impairment and conductive and mixed hearing impairment, such as the range highlighted in FIG. 7, a middle ear implant prosthetic hearing product such as the Vibrant Med-El Soundbridge® Hearing System can be beneficial. This may include potential patients who cannot use conventional hearing aids for medical reasons or who are dissatisfied with other hearing devices. This type of device includes an implanted Vibrating Ossicular Prosthesis (VORP) and an externally worn Audio Processor (AP). A very small transducer (Floating Mass Transducer, FMT) mechanically vibrates the middle ear structures to deliver audio frequency information up to 8,000 Hz.

Again, other additional selection criteria also may be taken into account to select such a product. For example, pure tone threshold indications for a hybrid electric acoustic product are a mild to moderate low frequency sensorineural hearing impairment, sloping to a profound hearing impairment in the higher frequencies, with speech word comprehension scores ≦60% at 65 dBSPL in the best aided condition. Examples of other additional selection criteria may include that the prospective patient have stable hearing thresholds, be hearing aid experienced, have relatively normal middle ear function as shown by audiometric thresholds, tympanometry and/or acoustic reflexes, and open-set word comprehension score ≦50% at the most comfortable listening level using head phones, or at 65 dB SPL in the free field using hearing aid(s). Other additional selection criteria may include that there be no skin conditions preventing attachment of the Audio Processor, realistic patient expectations, and absence of retrocochlear and central auditory disorders.

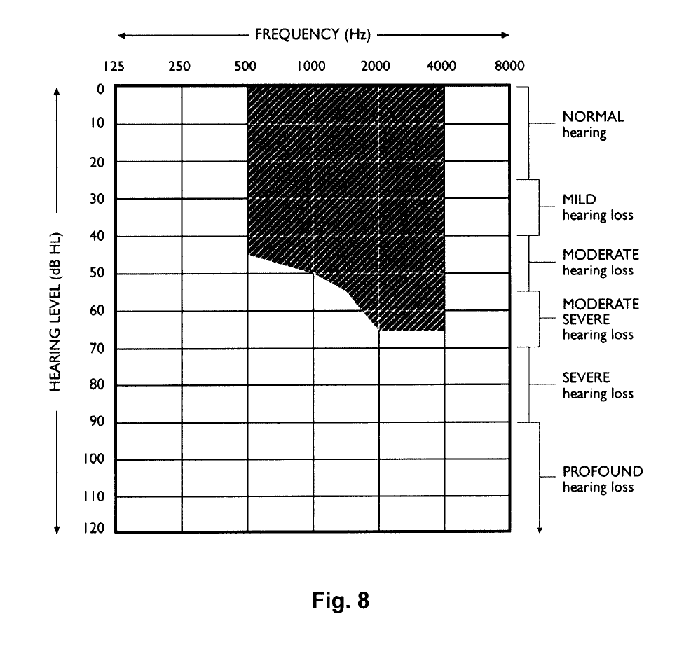

Similarly, for patient audiograms indicating conductive and mixed hearing impairment, selection criteria to have a middle ear implant product selected may include that bone-conduction thresholds are within the shaded region in FIG. 8, absence of active middle ear infection and/or chronic fluid in the ear, and stable bone conduction thresholds. Other additional selection criteria may include that there be no skin conditions preventing attachment of the Audio Processor, realistic patient expectations, and absence of retrocochlear and central auditory disorders.

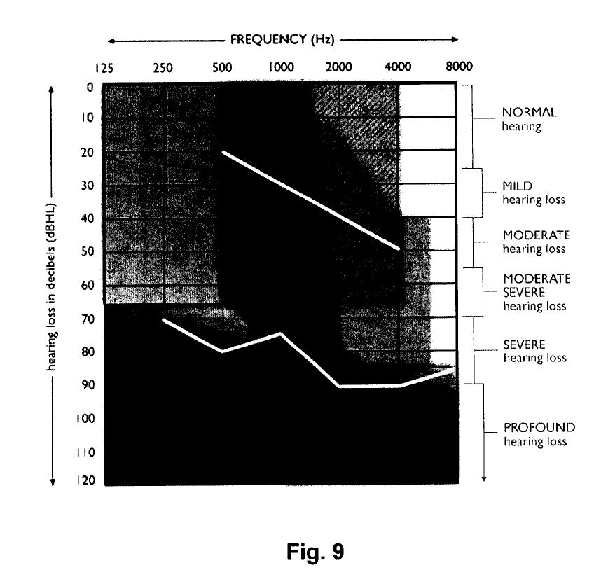

FIG. 9 shows an example of one specific interactive user interface for displaying a representation of the product selection process. The display shows a measured patient audiogram overlaying a display of the different product audiogram regions that are available, where the different product audiogram regions may coded by color, shading, and/or pattern. A specific prosthetic hearing product is selected where the associated product audiogram covers a significant portion of the patient audiogram.

Embodiments of the invention may be implemented in part using any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A system for selecting a prosthetic hearing product comprising:
a patient testing module for testing hearing characteristics of a potential patient to determine a patient audiogram representing patient hearing impairment;
a product comparison module for comparing the patient audiogram to a plurality of product audiograms, each product audiogram representing a region of hearing impairment treatable by a specific prosthetic hearing product; and
a product selection module for selecting a specific prosthetic hearing product for the potential patient based on the audiogram comparison.

2. A system according to claim 1, wherein the prosthetic hearing products include at least one cochlear implant system.

3. A system according to claim 2, wherein the at least one cochlear implant system includes a plurality of different electrode array options.

4. A system according to claim 2, wherein the at least one cochlear implant system includes a plurality of different external processor options.

5. A system according to claim 1, wherein the prosthetic hearing products include at least one hybrid electric acoustic implant system.

6. A system according to claim 1, wherein the prosthetic hearing products include at least one middle ear implant system.

7. A system according to claim 1, wherein the product selection module further considers additional selection criteria together with the audiograms for selecting a specific prosthetic hearing product.

8. A system according to claim 7, wherein the additional selection criteria includes at least one of past medical history of the potential patient, speech scores representing word comprehension testing results, speech scores representing syllable comprehension testing results, and an overall impairment value representing severity of hearing impairment in the potential patient.

9. A system according to claim 1, further comprising:
an interactive user interface for displaying a representation of the product selecting.

10. A system according to claim 9, wherein the interactive user interface displays the patient audiogram and at least one of the product audiograms.

11. A system according to claim 9, wherein a plurality of product audiograms are displayed.

12. A system according to claim 9, wherein the product audiograms are color coded.

13. A method for selecting a prosthetic hearing product comprising:
testing hearing characteristics of a potential patient to determine a patient audiogram representing patient hearing impairment;
comparing the patient audiogram to a plurality of product audiograms, each product audiogram representing a region of hearing impairment treatable by a specific prosthetic hearing product; and
selecting a specific prosthetic hearing product for the potential patient based on the audiogram comparison.

14. A method according to claim 13, wherein the prosthetic hearing products include at least one cochlear implant method.

15. A method according to claim 14, wherein the at least one cochlear implant method includes a plurality of different electrode array options.

16. A method according to claim 14, wherein the at least one cochlear implant method includes a plurality of different external processor options.

17. A method according to claim 13, wherein the prosthetic hearing products include at least one hybrid electric acoustic implant method.

18. A method according to claim 13, wherein the prosthetic hearing products include at least one middle ear implant method.

19. A method according to claim 13, wherein selecting a specific prosthetic hearing product includes considering additional selection criteria together with the audiograms.

20. A method according to claim 19, wherein the additional selection criteria includes at least one of past medical history of the potential patient, speech scores representing word comprehension testing results, speech scores representing syllable comprehension testing results, and an overall impairment value representing severity of hearing impairment in the potential patient.

21. A method according to claim 13, further comprising:
displaying an interactive representation of the comparing and selecting steps.

22. A method according to claim 21, wherein the interactive representation includes the patient audiogram and at least one of the product audiograms.

23. A method according to claim 21, wherein the interactive representation includes a plurality of product audiograms.

24. A method according to claim 21, wherein the product audiograms are color coded.

25. A computer program product in a computer readable storage medium for selecting a prosthetic hearing product, the computer program product comprising:
program code for testing hearing characteristics of a potential patient to determine a patient audiogram representing patient hearing impairment;
program code for comparing the patient audiogram to a plurality of product audiograms, each product audiogram representing a region of hearing impairment treatable by a specific prosthetic hearing product; and
program code for selecting a specific prosthetic hearing product for the potential patient based on the audiogram comparison.
